(19) United States
(12) Patent Application Publication
(10) Pub. No.: US 2022/0249071 A1
(43) Pub. Date: Aug. 11, 2022

(54) Endoscope Biopsy System and Method for Ductoscopy

(71) Applicant: The Board of Regents of the University of Oklahoma, Norman, OK (US)

(72) Inventors: William Dooley, Oklahoma City, OK (US); Apurba Mukherjee, The Village, OK (US)

(73) Assignee: The Board of Regents of the University of Oklahoma, Norman, OK (US)

(21) Appl. No.: 17/627,735

(22) PCT Filed: Jul. 17, 2020
(86) PCT No.: PCT/US2020/042637

§ 371 Date: Jan. 17, 2022

Related U.S. Application Data
(60) Provisional application No. 62/876,389, filed on Jul. 19, 2019.

(51) Int. Cl. A61B 10/00; A61B 1/313; A61B 1/018; A61B 10/04; A61B 1/015

(52) U.S. Cl. CPC A61B 10/0041; A61B 1/313; A61B 2010/0216; A61B 10/04; A61B 1/015; A61B 1/018

(57) ABSTRACT

An endoscope biopsy device has a hub, an insertion tube connected to the hub, an endoscope module connected to the hub, and a biopsy module connected to the hub. The endoscope module includes a fiber optic line extending through the hub and into the insertion tube. The biopsy module is configured to admit biopsy tools to the patient through the insertion tube from the biopsy module while simultaneously providing a visual examination of the portion of the patient from which the biopsy is taken. In some embodiments, the endoscope biopsy device is configured to perform a biopsy during a ductoscopy on a human patient.

### RELATED APPLICATIONS

This application claims the benefit of U.S. Provisional Patent Application Ser. No. 62/876,389 filed Jul. 19, 2019 entitled “Endoscope Biopsy System for Ductoscopy,” the disclosure of which is herein incorporated by reference.

### BACKGROUND

Breast cancer remains a significant health concern in countries with a high human development index (HDI). Incidence rates are rising in a number of countries, with non-hereditary, environmental and endogenous factors contributing to the growth of cancer in these areas. Currently, mammography screening is the standard for detecting breast cancer. Although widely accepted, mammography is expensive and unavailable in many of the regions where breast cancer rates are increasing. Other diagnostic measures, including palpation and ultrasound techniques, are incapable of reliably identifying early-stage cancer or precancerous cells.

Recently, micro endoscopes have been developed that permit the direct examination of breast tissue with fiber optic bundles. Typically, prior to or during the introduction of the endoscope, biopsy device, or other surgical object into the patient's body, an “introducer” pumps liquids into the small incision or natural opening that are used to enlarge the area surrounding the target surgical site in order allow for easier insertion, better visual inspection, and more work area for the surgeon.

The introducer typically includes a hub or rigid hollow vessel that is capable of containing low-pressure liquid there through a long rigid cannula or tube at its distal end and connected to an endoscope port and an insufflation port on its proximal end. The insufflation port is typically coupled to a flow source of gas or liquid that forces the pressurized flow of gas or liquid into and through the hub, into the cannula, and out the distal end of the cannula. With this flow of gas or liquid occurring, the distal end of the cannula is inserted into the patient's body to enlarge the targeted area of the patient's body. The flow of low-pressure liquid and ensuing enlargement of the targeted anatomy allows for easier insertion, navigation, maneuverability, and extraction of the micro endoscope. The endoscope is inserted into the endoscope port, through the hub, through the cannula, and then out of the distal end of the cannula into the patient's body.

The distal tip of the endoscope must protrude through the distal tip of the cannula on the introducer so that it can provide illumination and receive images from the patient's body. The distal tip of the endoscope must remain within a certain range or zone of extension beyond the distal tip of the cannula on the introducer for the endoscope to function properly.

U.S. Pat. No. 8,323,181 issued on Dec. 4, 2012 and entitled “Endoscope with Variable Incident Light and Laser Source Platform” discloses a micro endoscope that is designed to be inserted through an “introducer” into the patient's body for a direct examination of internal organs and tissues. The patent specifically describes a process for using the micro endoscope for examining milk ducts or other ductal structures or cavities accessible by 1mm maximal external diameter micro-endoscopes. The micro endoscope disclosed in this patent includes access device for ablation tools such as laser, RF, Microwave, and others that are capable of tissue ablation, tissue cutting, tissue resection or tissue shrinking.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function.

FIG. 1 is a side view of an endoscope biopsy device constructed in accordance with an exemplary embodiment.

FIG. 2 cross-sectional view of the insertion tube of the endoscope biopsy device of FIG. 1.

### DETAILED DESCRIPTION

Although micro endoscopes like the one disclosed in U.S. Pat. No. 8,323,181 represent a significant advancement in the art, there remains a need for a device that couples the advanced imaging functionality with the ability to perform ductoscopy procedures. The ductoscopy procedure involves inserting the fiber optic micro endoscope in the natural opening of the milk duct (or other ductal structures or cavities of similar small capacity) and looking for abnormal cells and tissues in the lining of the duct or cavity. It is known that in almost all cases, breast cancer originates in milk ducts. If abnormal tissues and cells are found, the patient must undergo invasive surgical procedures to extract the abnormal tissues and cells. There is, therefore, a need for an improved system and method for extracting abnormal cells and tissues at the time of the ductoscopy procedure while under direct vision from the physician. The present disclosure is directed at addressing these and other deficiencies in the prior art.

In some embodiments, the present disclosure is directed to an endoscope biopsy device for use on a patient. The endoscope biopsy device has a hub, an insertion tube connected to the hub, an endoscope module connected to the hub, and a biopsy module connected to the hub. The endoscope module includes a fiber optic line extending through the hub and into the insertion tube. The biopsy module is configured to admit biopsy tools to the patient through the insertion tube from the biopsy module.

In other embodiments, the present disclosure is directed to a method for conducting a breast ductoscopy procedure on a patient. The method begins with the step of providing an endoscope biopsy device that includes a hub, an insertion tube connected to the hub, an endoscope module, and a biopsy module. The method continues with the steps of inserting the insertion tube into an interior of a milk duct of the patient, illuminating the interior of the milk duct with a first fiber optic line extending from the endoscope module, and retrieving live images of the interior of the milk duct with a second fiber optic line extending from the endoscope module. The method continues with the steps of inserting a biopsy tool into the interior of the milk duct through the biopsy module, and retrieving a tissue sample from the interior of the milk duct with the biopsy tool while visually observing live images of the interior of the milk duct with the endoscope module.

Before describing various embodiments of the present disclosure in more detail by way of exemplary description, examples, and results, it is to be understood as noted above that the present disclosure is not limited in application to the details of methods and apparatus as set forth in the following description. The present disclosure is capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

As utilized in accordance with the methods and apparatus of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges (e.g., in units of length such as micrometers or millimeters) include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error. Further, in this detailed description, each numerical value (e.g., temperature or time) should be read once as modified by the term “about” (unless already expressly so modified), and then read again as not so modified unless otherwise indicated in context. As noted above, any range listed or described herein is intended to include, implicitly or explicitly, any number within the range, particularly all integers, including the end points, and is to be considered as having been so stated. For example, “a range from 1 to 10” is to be read as indicating each possible number, particularly integers, along the continuum between about 1 and about 10. Thus, even if specific data points within the range, or even no data points within the range, are explicitly identified or specifically referred to, it is to be understood that any data points within the range are to be considered to have been specified, and that the inventors possessed knowledge of the entire range and the points within the range. Unless otherwise stated, the term “about” or “approximately”, where used herein when referring to a measurable value such as an amount, length, thickness, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art.

As used herein, the term “substantially” means that the subsequently described parameter, event, or circumstance completely occurs or that the subsequently described parameter, event, or circumstance occurs to a great extent or degree. For example, the term “substantially” means that the subsequently described parameter, event, or circumstance occurs at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 90%, or at least 91%, or at least 92%, or at least 93%, or at least 94%, or at least 95%, or at least 96%, or at least 97%, or at least 98%, or at least 99%, of the referenced dimension or measurement (e.g., length).

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Where applicable, although state diagrams, flow diagrams or both may be used to describe embodiments, the present disclosure is not limited to those diagrams or to the corresponding descriptions. For example, flow need not move through each illustrated box or state, or in exactly the same order as illustrated and described. Methods of the present disclosure may be implemented by performing or completing manually, automatically, or a combination thereof, selected steps or tasks. The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility). Still further, additional aspects of the various embodiments of the instant disclosure may be found in one or more appendices attached hereto and/or filed herewith, the disclosures of which are incorporated herein by reference as if fully set out at this point.

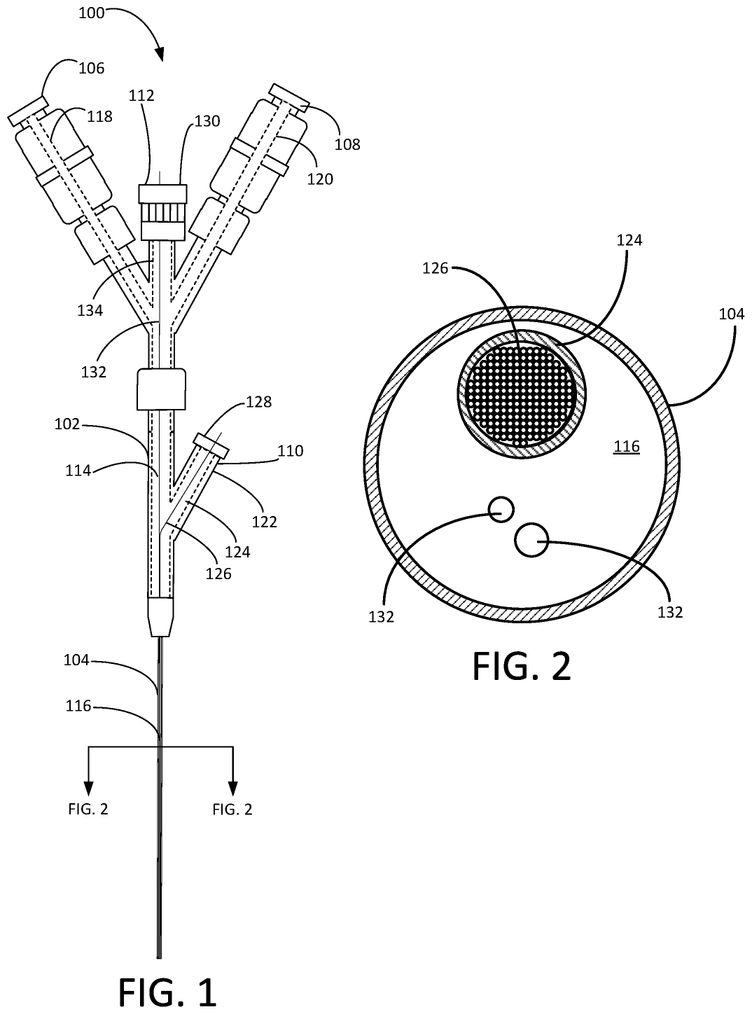

The present disclosure is therefore directed, in non-limiting embodiments, to an endoscope biopsy device 100 configured to perform endoscopic procedures, including breast ductoscopy procedures. Referring to FIG. 1, shown therein is a side view of the endoscope biopsy device 100. The endoscope biopsy device 100 includes a hub 102, an insertion tube 104, a fluid inlet 106, a fluid outlet 108, an endoscope module 110 and a biopsy module 112. The hub 102 includes a central hub channel 114 that extends along the longitudinal axis of the hub 102. Similarly, the insertion tube 104 is hollow and includes an insertion tube channel 116 that connects to the central hub channel 114. The insertion tube 104 and hub 102 can be constructed as a unitary component or as two separate components joined by mechanical means, such as a threaded or push-fit connection at the interface between the hub 102 and the insertion tube 104. In some embodiments, the insertion tube 104 has an outer diameter that is less than about 1 mm.

The hub 102 includes a fluid inlet channel 118 and a fluid outlet channel 120 that connect the fluid inlet 106 and fluid outlet 108 to the central hub channel 114. The fluid inlet 106 and fluid outlet 108 may each include a Luer lock or similar common connection adaptor. During use, fluids such as saline liquid and sterile water can be passed into the hub 102 from the fluid inlet 106. The fluids pass through the central hub channel 114 into the insertion tube channel 116, where the fluids can be used to irrigate and insufflate the patient's targeted anatomy. In particular, insufflating milk ducts during a ductoscopy facilitates viewing and biopsy procedures. The fluids can be evacuated from the patient by draining the fluids through the fluid outlet 108. In some applications, the fluid outlet 108 can also be used to retrieve tools and tissue samples.

The endoscope module 110 includes an endoscope housing 122 connected to the hub 102 and an endoscope channel 124 that extends from the housing 122, through the central hub channel 114 and into the insertion tube channel 116. A fiber optic line 126 can be passed through the endoscope channel 124. In exemplary embodiments, the fiber optic line 126 includes a bundle of optically active fibers that conduct light to and from the patient. The endoscope module 110 includes an endoscope inlet port 128 that can be used to pass the fiber optic line 126 into the endoscope channel 124 and to connect the fiber optic line 126 to viewing equipment, light sources and laser sources. The endoscope inlet port 128 can be a Luer lock or similar common connection adaptor. The endoscope module 110 permits the introduction of the fiber optic line 126 into the endoscope channel 124 within the central hub channel 114 and insertion tube channel 116. In some embodiments, viewing lenses and image sensors are incorporated into the endoscope module 110.

As explained in U.S. Pat. No. 8,323,181, the entire disclosure of which is herein incorporated by reference, the fiber optic line 126 may include about 3,000 to 10,000 continuous optical fibers that together form an image bundle that is about 0.3 to about 0.7 millimeters in outer diameter. A first group of the optical fibers are used to carry images from the patient's targeted anatomy back to the endoscope module for viewing. A second group of the optical fibers are used to carry light to the patient's targeted anatomy to illuminate and contrast the tissues under investigation.

The biopsy module 132 is connected to the hub 102 and includes a biopsy port 130 that selectively permits the introduction of biopsy tools 132 through a biopsy channel 134 into the adjacent central hub channel 114. The biopsy tools 132 can be manipulated and retrieved through the biopsy port 130. The biopsy port 130 can be a hydrophobic filter cap or similar connection adapter that prevents fluid from exiting through the biopsy port 130. The biopsy tools 132 may include micro brushes, wire loops or other miniaturized tools that are configured to retrieve tissue samples from the patient's targeted anatomy. In some applications, the biopsy tools 132 can be used to deploy contrast dyes, coil springs, stents, medicines, treatment modalities, or other devices into the patient's targeted anatomy for tracking tissue changes, for immediate imaging, or for therapeutic purposes.

FIG. 2 provides a cross-sectional view of the insertion tube channel 116. The biopsy tools 132 and endoscope channel 124 are contained within the insertion tube channel 116 of the insertion tube 104. The bundle of discrete fibers is visible within the fiber optic line 126 within the endoscope channel 124. In some embodiments, the endoscope channel 124 has an inner diameter of about 0.8 mm.

During use of the endoscope biopsy device 100 during a ductoscopy, the insertion tube 104 can be placed through an introducer into the patient's dilated and insufflated milk duct. In some embodiments, the fiber optic line 126 and biopsy tools 132 are installed within the hub 102 and insertion tube 104 before the insertion tube 104 is inserted into the patient's milk duct. In other embodiments, the insertion tube 104 is inserted into the patient's milk duct before the fiber optic line 126 and biopsy tools 132 are introduced through the hub 102. In either case, the fiber optic line 126 can be activated to illuminate the anatomy within the milk duct and to retrieve live images of the tissue inside the milk duct under direct vision.

The endoscope biopsy device 100 also permits the simultaneous retrieval of tissue samples using the biopsy tools 132. The biopsy tools 132 can be inserted through the biopsy channel 134, central hub channel 114 and insertion tube channel 116 into the patient's milk duct, where abnormal tissue samples and ductal fluid can be retrieved while visually observing the targeted milk duct tissues. The ability to obtain samples of abnormal tissues during direct visual observation presents a significant advantage over the prior art and permits the potential detection of breast cancer at very early stages. Additionally, the fluids evacuated from the fluid outlet 108 can be presented for pathological study to determine if abnormalities exist in the patient's nipple fluids.

Although the endoscope biopsy device 100 is well suited for use in conducting breast tissue examinations, the endoscope biopsy device 100 will also find utility in other minimally invasive investigations. The small size of the insertion tube 104 permits use of the endoscope biopsy device 100 in numerous clinical applications, including salivary and bile duct examinations. Any body cavity that can be accessed by a standard 20 G needle could similarly be accessed by the endoscope biopsy device 100.

Thus, the embodiments of the present disclosure are well adapted to carry out the objects and attain the ends and advantages mentioned above as well as those inherent therein. While the inventive device and system have been described and illustrated herein by reference to particular non-limiting embodiments in relation to the drawings attached thereto, various changes and further modifications, apart from those shown or suggested herein, may be made therein by those of ordinary skill in the art, without departing from the spirit of the inventive concepts.

What is claimed is:

1. An endoscope biopsy device for use on a patient, the endoscope biopsy device comprising:
a hub;
an insertion tube connected to the hub;
an endoscope module connected to the hub, wherein the endoscope module comprises a fiber optic line extending through the hub and into the insertion tube; and
a biopsy module connected to the hub, wherein the biopsy module is configured to admit biopsy tools to the patient through the insertion tube from the biopsy module.

2. The endoscope biopsy device of claim 1, wherein the hub comprises:
a central hub channel;
a fluid inlet in communication with the central hub channel; and
a fluid outlet in communication with the central hub channel.

3. The endoscope biopsy device of claim 2, wherein the insertion tube comprises an insertion tube channel in communication with the central hub channel.

4. The endoscope biopsy device of claim 2, wherein the endoscope module further comprises:
an endoscope housing; and
an endoscope channel extending through the endoscope housing to the central hub channel.

5. The endoscope biopsy device of claim 2, wherein the biopsy module comprises:
a biopsy port; and
a biopsy channel extending through the biopsy port to the central hub channel.

6. The endoscope biopsy device of claim 5, wherein the biopsy port comprises a hydrophobic filter cap.

7. The endoscope biopsy device of claim 6, wherein the biopsy tools are miniaturized tools selected from the group consisting of brushes and wire loops configured to retrieve tissue samples from the patient.

8. The endoscope biopsy device of claim 6, wherein the biopsy tools are miniaturized tools configured to deploy fluids into the patient.

9. The endoscope biopsy device of claim 1, wherein the patient is a human patient.

10. A method for conducting a breast ductoscopy procedure on a patient, the method comprising the steps of:
providing an endoscope biopsy device that includes a hub, an insertion tube connected to the hub, an endoscope module, and a biopsy module;
inserting the insertion tube into an interior of a milk duct of the patient;
illuminating the interior of the milk duct with a first fiber optic line extending from the endoscope module;
retrieving live images of the interior of the milk duct with a second fiber optic line extending from the endoscope module;
inserting a biopsy tool into the interior of the milk duct through the biopsy module; and
retrieving a tissue sample from the interior of the milk duct with the biopsy tool while visually observing live images of the interior of the milk duct with the endoscope module.

11. The method of claim 10, wherein the step of inserting the insertion tube further comprises inserting the insertion tube through an introducer into the interior of the milk duct of the patient.

12. The method of claim 10, further comprising the step of circulating a fluid from the endoscope biopsy device into the interior of the milk duct of the patient.

13. The method of claim 12, wherein the step of circulating a fluid from the endoscope biopsy device comprises:
injecting a fluid into the interior of the milk duct through the insertion tube from a fluid inlet of the endoscope biopsy device; and
removing fluid from the interior of the milk duct through the insertion tube with a fluid outlet of the endoscope biopsy device.

14. The method of claim 10, further comprising the step of injecting a dye into the interior of the milk duct.

15. The method of claim 10, further comprising the step of injecting a medicinal fluid into the interior of the milk duct.

16. An endoscope biopsy device for use on a patient, the endoscope biopsy device comprising:
a hub;
an insertion tube connected to the hub;
an endoscope module connected to the hub, wherein the endoscope module comprises:
an endoscope housing;
an endoscope channel extending through the endoscope housing to the central hub; and
a fiber optic line extending through the hub and into the insertion tube; and
a biopsy module connected to the hub, wherein the biopsy module is configured to admit biopsy tools through the insertion tube from the biopsy module.

17. The endoscope biopsy device of claim 16, wherein the hub comprises:
a central hub channel;
a fluid inlet in communication with the central hub channel; and
a fluid outlet in communication with the central hub channel.

18. The endoscope biopsy device of claim 16, wherein the biopsy module comprises:
a biopsy port; and
a biopsy channel extending through the biopsy port to the central hub channel.

19. The endoscope biopsy device of claim 18, wherein the biopsy tools are miniaturized tools selected from the group consisting of brushes and wire loops configured to retrieve tissue samples from the patient.

20. The endoscope biopsy device of claim 18, wherein the biopsy tools are miniaturized tools configured to deploy fluids into the patient.
